(19) United States
(12) Patent Application Publication
(10) Pub. No.: US 2008/0033310 A1
(43) Pub. Date: Feb. 7, 2008

(54) METHOD AND APPARATUS FOR ARTERIAL BLOOD PRESSURE MEASUREMENT AND INDIVIDUALIZED RECTIFYING TECHNOLOGY USING THIS METHOD

(76) Inventors: Meng-su YU, Beijing (CN); Hai-Yan Xiang, Beijing (CN); Zu-Iai Tao, Beijing (CN)

Correspondence Address: Michael L. Crapenhoft;c/o Hiroe and Associates, 4-3 Usa 3-chome, Gifu City, 500-8368, JP

(21) Appl. No.: 11/775,870

(22) Filed: Jul. 11, 2007

(30) Foreign Application Priority Data
May 24, 2005 (CN) 200510071813.0

(51) Int. Cl. A61B 5/0225

(52) U.S. Cl. 600/493

(57) ABSTRACT

A method and an apparatus for indirect, quantitative estimation of beat-to-beat arterial blood pressure utilizing individualized rectifying technology. A function of T_{K}=H(P) that describes the relationship between Korotkoff's sound delay time Tₖ and cuff pressure P is obtained by measuring different cuff pressures P and the corresponding Korotkoff's sound delay times Tₖ in a Korotkoff's sound sensor that is distal to the cuff. Keeping the cuff pressure at a constant value Pₘ, the blood pressure change can be calculated by using the Korotkoff's sound delay time Tₖₘ according to the function of Tₖ=H(P). The invention can measure beat-to-beat arterial blood pressures indirectly. The technology can be applied to obtain individualized coefficients of a regress equation for continuous arterial blood pressure measurement by the instantaneous blood pressure fluctuation, and the technology makes the rectifying technique more safe, effective, and less erroneous and makes the operation of noninvasive continuous blood pressure measurement for long times more practical.

### FIELD OF THE INVENTION

The present invention relates to both a method and an apparatus for noninvasive arterial blood pressure measurement and individualized rectifying technology using the method for continuous blood pressure measurement.

### BACKGROUND OF THE INVENTION

Noninvasive blood pressure (BP) measurement is the technology to measure the BP indirectly by the parameter detection of an arterial vessel wall's beat or arterial volume. There are two types of noninvasive BP measurement: intermissive measurement and continuous measurement. Intermissive measurement can get the value of BP at specific time points, but due to the consistent change of BP on the arterial vessel wall at every heart beat and every time point, the systolic pressure and the diastolic pressure may not represent a meaningful value for a given subject, and these two values relate to different heart beats. Continuous measurement technology, which measures the BP without intermission, can provide the beat-to-beat BP or a continuous arterial BP wave. It is very important to realize noninvasive continuous BP measurement. But, until now, there is not an ideal measurement method that can achieve that aim.

BP measurement according to the pulse wave transit velocity (PWTV) is a type of noninvasive continuous BP measurement method. In 1922, Bazzett discovered that the PWTV or pulse wave transit time (PWTT) relates to arterial blood pressure, and additionally relates to arterial volume and the arterial vessel wall's flexibility. In 1957, Lansdown pointed that PWTT and arterial BP present a linear relationship to some extent and that this relationship is stable for a given subject in a period of time. Moreover, the coefficients that describe the linear relationship between PWTT and BP vary widely for different subjects with different arterial vessel tissue structures. But in past studies, the BP according to PWTT of different subjects was generally evaluated through the same coefficients, so the results can be distorted by errors.

An equation describing the relationship between beat-to-beat BP and PWTT for a given subject can be deduced based on the linear relationship between them:
BP=a+b×PWTT (A)
wherein, BP is arterial blood pressure, PWTT is pulse wave transit time, a and b are regressing coefficients to be estimated and which vary in different subjects, but for which in a given subject and over a short period, they are stable. Previous analysis shows that to evaluate arterial BP, the coefficient of a and b for a given subject must be obtained firstly, and after that, continuous arterial BP for a given subject can be computed by the continuous detection of PWTT (or PWTV). The coefficient a and b need to be rectified by means of individualized regressing technology, so that the value of continuous arterial BP computed by the regressing equation (A) can fit individual conditions well during the continuous detection of a pulse wave.

In principle, evaluating two pending parameters requires two groups of independent experimental data. PWTT and mean arterial pressure in the quiet condition for a given subject can be obtained, so coefficient a, i.e., the intercept, is easy to determine. Coefficient b=ΔBP/ΔPWTT, i.e., the slope, is always estimated by altering BP to get two groups of data. In order to change BP, exercise or drugs were often involved in the experiment, which can change the artery character and violate the premise that in a short period the linear relationship in equation (A) is consistent.

Yu Mengsun also believed that when body posture changed (for example, between supine and when elevating a leg), PWTT in the elevated leg would change. It is because the change of body posture alters the pressure in some vessels and then makes PWTV different from that of the normal state. If experimental data in a normal status and a posture changing status can be obtained, the coefficients a and b can be estimated from these data. This method can rectify parameters more accurately, but multi-group information relating to the beat-to-beat BP cannot be continuously gotten with the body posture changed.

### SUMMARY OF THE INVENTION

The purpose of the present invention is to offer a method and an apparatus that can measure the beat-to-beat arterial blood pressure from the information of arterial blood pressure. The invention also relates to an individualized rectifying technology which makes the blood pressure estimated by using continuous pulse wave measurement correspond to the individual reality.

To resolve the above problems, the invention uses a method to measure the arterial blood pressure:

(1) Wrapping the cuff around the trunk or limb of the subject; getting a series of different values of cuff pressure P and the corresponding Korotkoff's sound delay times T_{K}; getting a functional relation of T_{K}=H(P) between Korotkoff's sound delay time and cuff pressure.

(2) Calculating Korotkoff's sound delay time (T_{K}m) under the corresponding cuff pressure (Pm); according to the fact that the change of delay time caused by change of cuff pressure is approximately equal in magnitude and inverse in direction compared with the change caused by blood pressure and using the equation relation of T_{K}=H(P) between Korotkoff's sound delay time T_{K} and cuff pressure P, we can estimate the change of blood pressure corresponding to the detected Korotkoff's sound delay time.

The Korotkoff's sound delay time mentioned above is the time that the Korotkoff's sound arrived from the fixed reference point to the corresponding cycle of a heartbeat. Said fixed reference point can be the ECGR wave peak (FIG. 1), or the ascending edge of the pulse wave in the cuff (FIG. 2).

The equipment to implement the above method comprises: the cuff, the inflating unit and deflating unit for the cuff the cuff pressure sensor, the Korotkoff's sound sensor, and the ECG electrode. The output signal ports of the cuff pressure sensor and Korotkoff's sound sensor are connected to the microprocessor through the signal conditioning circuit. The ECG electrode is connected to the microprocessor via an ECG circuit, and the microprocessor has a printing output and/or data display equipment.

The individualized rectifying technology according to said method is:

Constructing a regress equation between a pulse wave transit time (PWTT) and beat-to-beat arterial blood pressure (BP),
BP=a+b×PWTT (A)

In the equation, BP is the arterial blood pressure, PWTT is the pulse wave transit time corresponding to the BP, and the parameters a and b are the regress coefficients. After individualized rectification of the parameters a and b, based on the continuous measurement of pulse wave transit time and using the above equation, we can estimate the continuous change of the individual blood pressure for a given subject, characterized in that the individualized rectifying method for b is:

(1) Wrapping the cuff around the trunk or limb of the given subject, getting a series of different values of cuff pressure P and the corresponding Korotkoff's sound delay times T_{K}; then we can get the function relation of T_{K}=H(P) between Korotkoff's sound delay time and cuff pressure.

(2) Calculating Korotkoff's sound delay time under the corresponding cuff pressure Pₘ, using the said function relation of T_{K}=H(P) between Korotkoff's sound delay time and cuff pressure, we can estimate the change value of blood pressure corresponding to the Korotkoff's sound delay time.

(3) Recording the pulse wave transit times corresponding to the Korotkoff's sound delay time in step 2.

(4) Based on the data measured in step 2 and 3, calculating the proportional coefficient between the change of mean arterial pressure ΔBP and the change of pulse wave transit time ΔPWTT, then we can obtain individualized rectified parameter b.

In step 2 of the individualized rectifying method, the mean arterial pressure Pm is preferred for said corresponding cuff pressure.

A further method of individualized rectifying technology is: during the measurement of the Korotkoff's sound delay time based on the corresponding cuff pressure in step 2, and by directing the subject's own behavior that can alter the blood pressure of the subject but which does not change the characteristic of the vascular wall, we can enhance the range of blood pressure change between different measurement points.

Further, the behavior that can alter the blood pressure of the subject is a deep breath.

The above-mentioned equation constructed for the subject is a regress equation between the PWTT and beat-to-beat arterial BP. The method of the present invention is the same with the equation between PWTV (pulse wave transit velocity) and beat-to-beat arterial BP, or other linear regress equations that have different performance details but have the same essence.

The present invention is designed based on the study about the relationship between Korotkoff's sound delay time T_{K}, the cuff pressure P, and arterial blood pressure BP. The following is the introduction of the invention's principle.

When measuring blood pressure by the conventional stethoscope method (also called Korotkoff's sound method), we firstly inflate the cuff until the cuff pressure exceeds systolic blood pressure, when the artery is impacted and shut off so that there is no blood flow in the artery. Then while deflating the cuff slowly, referring to FIG. 1, when the cuff pressure is somewhat under the systolic blood pressure, the first Korotkoff's sound corresponding to the time when the artery begins to open appears. Based on experiment, we discover the following rule: in a series of times when the artery opens, the interval T₁ from the first Korotkoff's sound to the R wave in the electrocardiogram is the longest, while the intervals T₂, T₃ . . . from the subsequent Korotkoff's sounds to the R wave in the electrocardiogram, are shorter and shorter respectively, and the shortest one appears at the time of the last Korotkoff's sound. Referring to FIG. 2, if the rising point of the pulse wave inside the cuff is used for the reference point, then Korotkoff's sound delay time T_{K} can also be defined as the interval from the rising point of the pulse wave inside the cuff to the appearance of the Korotkoff's sound. Similarly, as the cuff pressure P decreases, Korotkoff's sound delay times T₁, T₂, T₃ . . . with each heartbeat cycle becoming shorter and shorter.

To analyze the principle of the phenomenon, we find that the pressure change inside the artery is a gradual process rather than a sudden or abrupt rising. So with the decreasing of the pressure inside the cuff, within each heartbeat cycle, the earlier the artery opens, the earlier Korotkoff's sound appears. And also according to the fixed reference point (such as the R wave in an electrocardiogram or the rising point of the pulse wave inside the cuff or some other fixed reference point) in each corresponding cycle, Korotkoff's sound delay times are shorter and shorter.

We can draw a conclusion that Korotkoff's sound delay times decrease gradually with the dropping of the cuff pressure, so the functional relationship which is formed from a series of Korotkoff's sound delay times T_{K} and their corresponding cuff pressure P (referring to FIG. 3) in an overall deflating process can be drawn as a line approximation (referring to FIG. 4, in which L1 is a simple line approximation, and L2 is a quadratic line approximation) that indicates the changes of Korotkoff's sound delay times T_{K} with the corresponding cuff pressure P.

Additionally, FIG. 5 shows the line approximation of the T_{K}=H(P) corresponding to different blood pressure levels. In the figure, the line approximation corresponding to the higher blood pressure L2 sits to the left of the line approximation corresponding to the lower blood pressure L1. So we can know that, at the same level of cuff pressure, the Korotkoff's sound delay time corresponding to the higher blood pressure is lower than the Korotkoff's sound delay time corresponding to the lower blood pressure, and at different levels of blood pressure, the changes in Korotkoff's sound delay times corresponding to a unit cuff pressure change dT_{K}/dP are different as well.

The above-mentioned functional relationship of T_{K}=H(P) is obtained when cuff pressure is descending while BP is stable; at the same time, we observe that if cuff pressure is at a constant pressure between systolic blood pressure and diastolic blood pressure, pressure change inside the artery will result in the change of artery transmural pressure, and consequently Korotkoff's sound delay time will change. It can be considered that the change of Korotkoff's sound delay time induced by cuff pressure changes when blood pressure is stable is the same in size and inverse in direction of that induced by blood pressure change when cuff pressure is stable.

According to the above-mentioned rule, the present invention can estimate the blood pressure change of each heartbeat cycle corresponding to each Korotkoff's sound by detecting the Korotkoff's sound delay time under a specific cuff pressure. The method can also evaluate the blood pressure change of each beat.

The above-mentioned individualized rectifying technology of a continuous arterial blood pressure monitoring regress equation is based on the finding that the corresponding change of arterial blood pressure can be estimated by the Korotkoff's sound delay time. The following is the principle of this technology:

Supposing the regress equation between pulse wave transit time PWTT and beat-to-beat arterial blood pressure BP is:
BP=a+b×PWTT (A)

Before using PWTT to measure BP, the parameters a and b for a given subject must be calculated first. With some technologies, the subject's mean arterial blood pressure BP₀ and corresponding pulse wave transit time PWTT₀ can be measured, so the parameter a will be easy to get if the parameter b has been gotten. With two groups of blood pressure values and PWTT values at two different blood pressure levels, there must be two factors to obtain the parameter b, that is:

1. To change the value of the BP; and

2. To detect the change of the BP.

In fact, a human's BP is changing at any moment. But the instantaneous change of BP can't be measured non-invasively with the known technology, so the parameters can't be rectified by spontaneous BP change. The present invention can estimate the change of BP per beat based on the Korotkoff's sound delay time, and measure individualized rectifying parameters by using instantaneous changes of BP.

Otherwise, the change of BP and the change of the corresponding PWTT are so small in a quiet state that the error of calculation will inescapably increase. In order to increase the Signal-to-Noise Ratio (SNR) and get the bigger instantaneous change of BP, the prior project of this invention also tries to control breath or some other actions to alter the subject's BP.

The work process of the present invention's apparatus is: the cuff pressure P can be gradually increased or decreased through an inflation unit. In this process, a Korotkoff's sound sensor measures the arrival time of the sound and sends it to the CPU. At the same time, a heart beat signal is also sent to the CPU through electrocardiogram electrodes and an electrocardiogram detecting circuit. Thereby the value of time interval T_{K} that is from the fixed reference point of every heart beat cycle to the Korotkoff's start-point in the same cycle can be gotten, and the function of T_{K}=H(P) in the present invention can be obtained. Keeping the cuff pressure at a fixed value through a cuff pressure controller, measuring Korotkoff's sound delay time T_{Km} at the cuff pressure and using the function of T_{K}=H(P) to get the blood pressure change in the current heart cycle compared to the initial measurement (when the function of T_{K}=H(P) was obtained).

The method and apparatus in the present invention can provide beat-to-beat arterial blood pressure estimation, and create a new way for individualized rectifying technology of a continuous arterial blood pressure monitoring regress equation. This technology can use an instantaneous change of BP to get individualized rectifying parameters, and it can increase the possibility of technical realization for long-time noninvasive continuous blood pressure monitoring, with many merits such as safety, availability, less error and briefness.

The present invention applies deep breath to enhance the range of blood pressure changes of a subject in individualized rectifying method, which can improve the accuracy of rectification and reduce the errors and which is safe and reliable for the subject.

### BRIEF DESCRIPTION OF THE DRAWINGS

FIG. 1 is a diagram for the Korotkoff's sound delay time T_{K} with an ECG R wave as a reference point when cuff pressure decreases.

FIG. 2 is a diagram for the Korotkoff's sound delay time T_{K} with a cuff pulse wave's rising point as a reference point when cuff pressure decreases.

FIG. 3 shows the functional relationship between Korotkoff's sound delay time T_{K} and cuff pressure P.

FIG. 4 is a simple line approximation and a quadratic line approximation of the Korotkoff's sound delay time T_{K} and cuff pressure P.

FIG. 5 is a line approximation of the Korotkoff's sound delay time T_{K} and cuff pressure P at different blood pressure levels.

FIG. 6 shows the relationship between dT_{K}/dP and cuff pressure P.

FIG. 7 shows the process for acquiring and processing data in embodiment 2 of the invention.

FIG. 8 shows the relationship between Korotkoff's sound delay time T_{K} and cuff pressure P in embodiment 1, and shows the quadratic line approximation of T_{K}=H(P) in embodiment 1.

FIG. 9 shows the relationship between dT_{K}/dP and cuff pressure P according to the line approximation T_{K}=H(P) in FIG. 8.

FIG. 10 shows the process of calculating regress coefficient b in embodiment 3.

FIG. 11 is a block diagram of apparatus for arterial blood pressure measurement.

FIG. 12 is a block diagram of an embodiment of arterial blood pressure measurement apparatus.

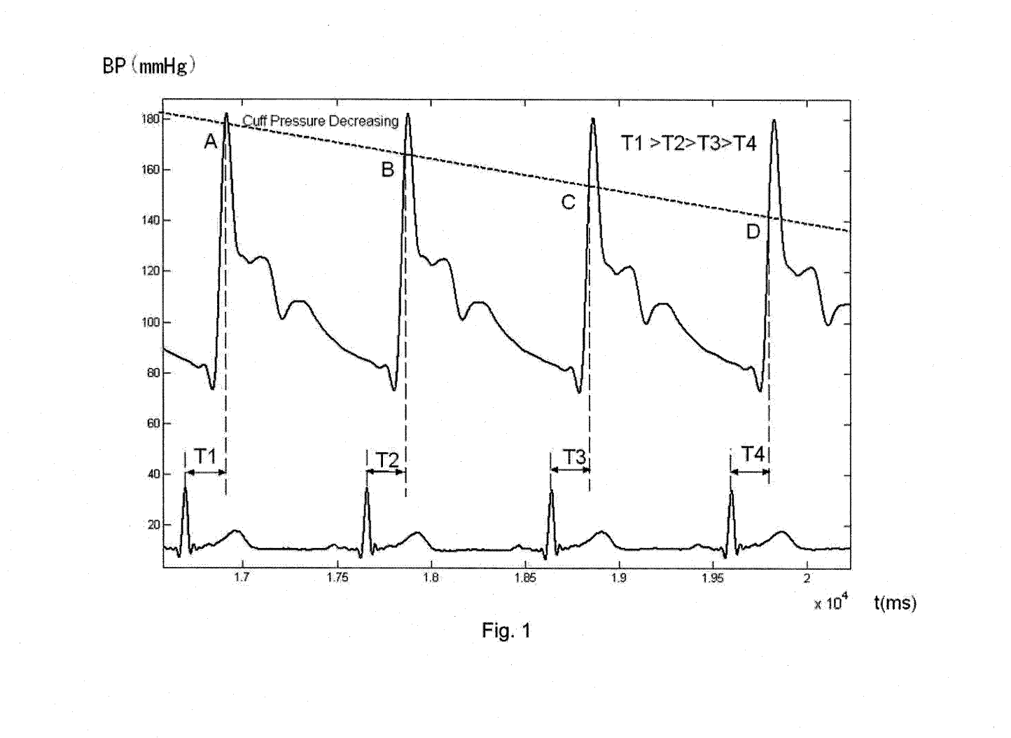

### DETAILED DESCRIPTION OF THE EMBODIMENTS

### EMBODIMENT 1

The following is about how to measure the arterial BP value of a certain heartbeat:

1. Wrapping the cuff around a subject's upper arm, obtaining a mean blood pressure value BP₀ by an oscillometric or an auscultatory method, and measuring the corresponding pulse wave transit time value PWTT₀ simultaneously.

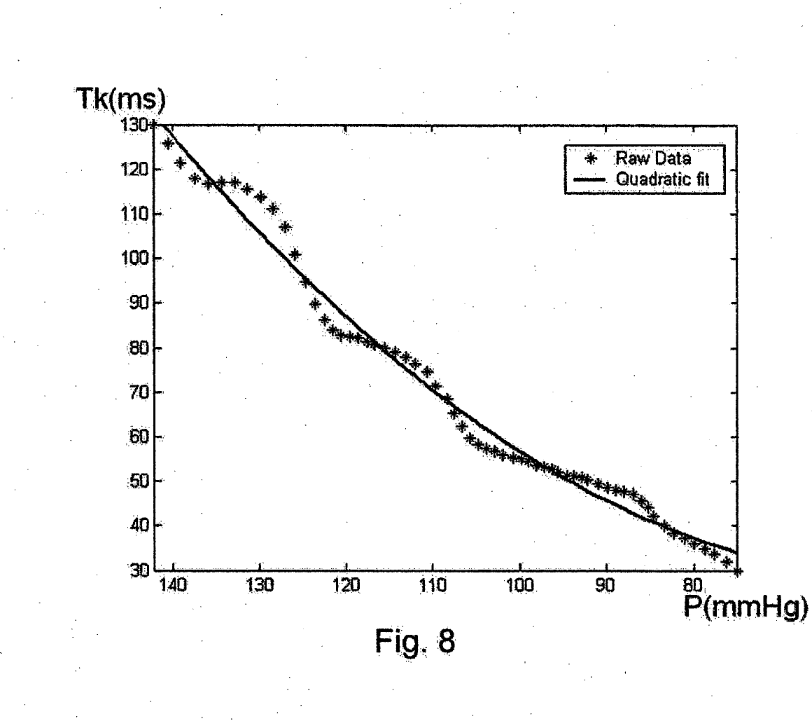

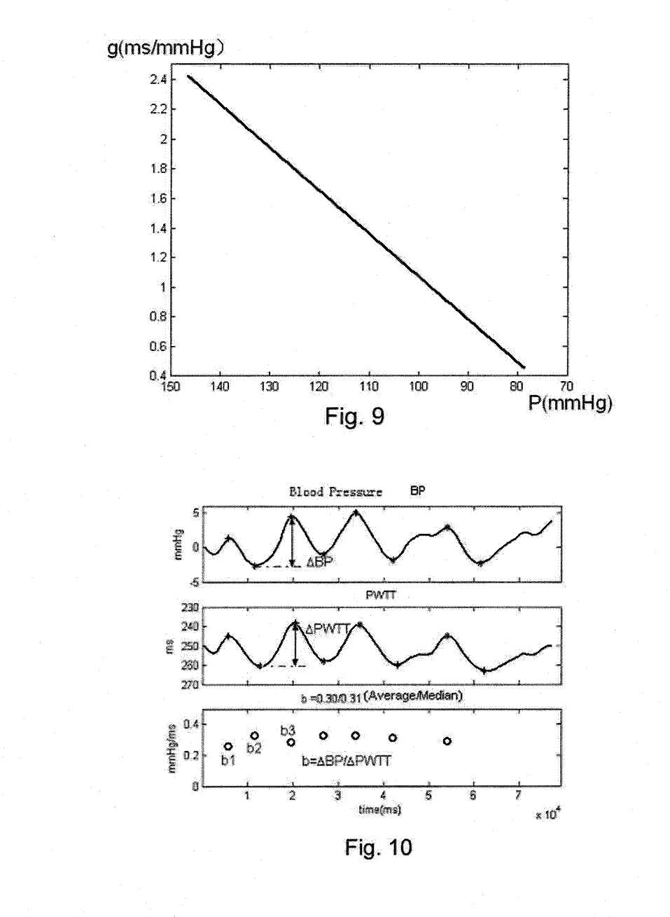

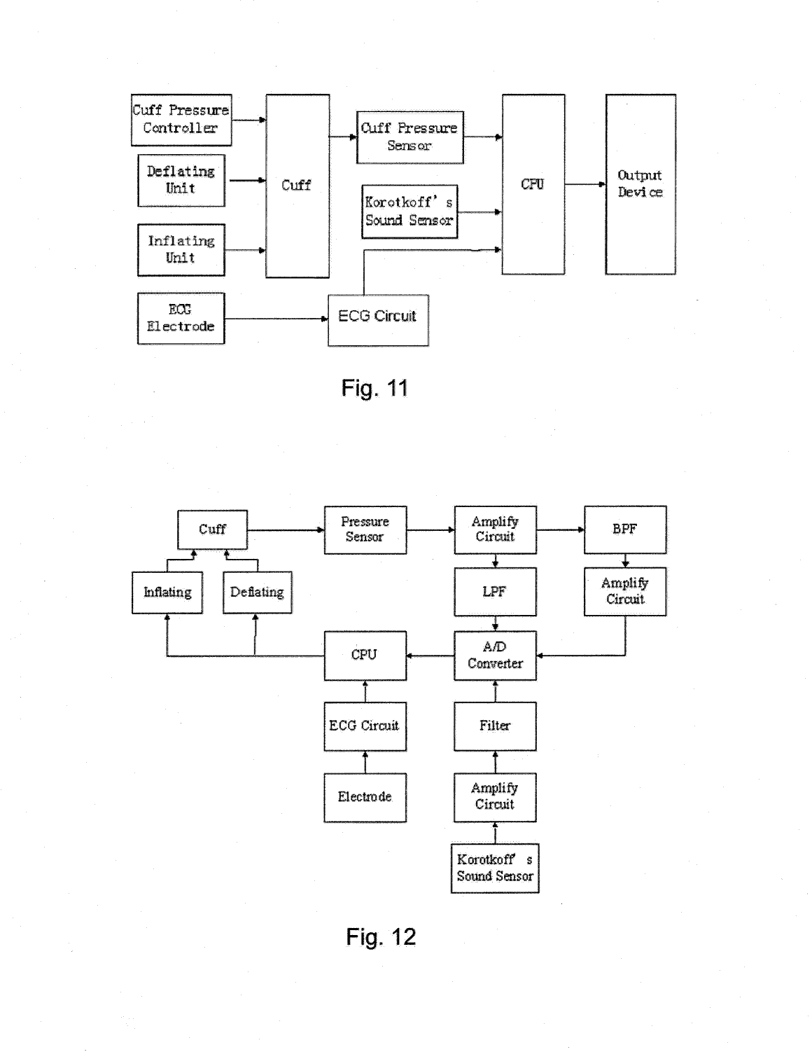

2. Recording a series of Korotkoff's sound delay time values T_{K} and the corresponding cuff pressure values P during deflation of the cuff, then constructing the function of T_{K}=H(P) between the Korotkoff's sound delay time and the cuff pressure value when the subject's mean blood pressure is at the level of BP₀. Obtaining the curve (FIG. 8) in which the Korotkoff's sound delay time is shortening as the cuff pressure decreases after quadratic line approximation of these discrete data. And according to the function of T_{K}=H(P), the function of g(P) between the dT_{K}/dP value of each point and the cuff pressure is obtained too. As shown in FIG. 9, (g(P)=dT_{K}/dP).

Based on these said individualized functions, the following data can be measured and calculated.

3. Measuring Korotkoff's sound delay time value T_{Km} when the cuff pressure is at a certain known value Pm that is between systolic blood pressure SBP and diastolic blood pressure DBP. Based on the function of T_{K}=H(P), obtaining Korotkoff's sound delay time T_{Km0} when the cuff pressure value is equal to Pₘ, and calculating the difference ΔT_{Km} between T_{Km} and T_{Km0}.

4. Calculating the g(Pₘ) value when the cuff pressure value is equal to Pₘ, according to the said function of g(P), in which the dT_{K}/dP of each point changes with the cuff pressure value P.

5. Based on the equation: g(Pₘ)=ΔT_{Km}/ΔBPₘ, obtaining the BP change value ΔBPₘ corresponding to the Korotkoff's sound delay time value T_{Km}.

The blood pressure value of this beat is equal to the summation of the BP change ΔBPₘ and the mean blood pressure value BP₀ that is used to establish the function of T_{K}=H(P).

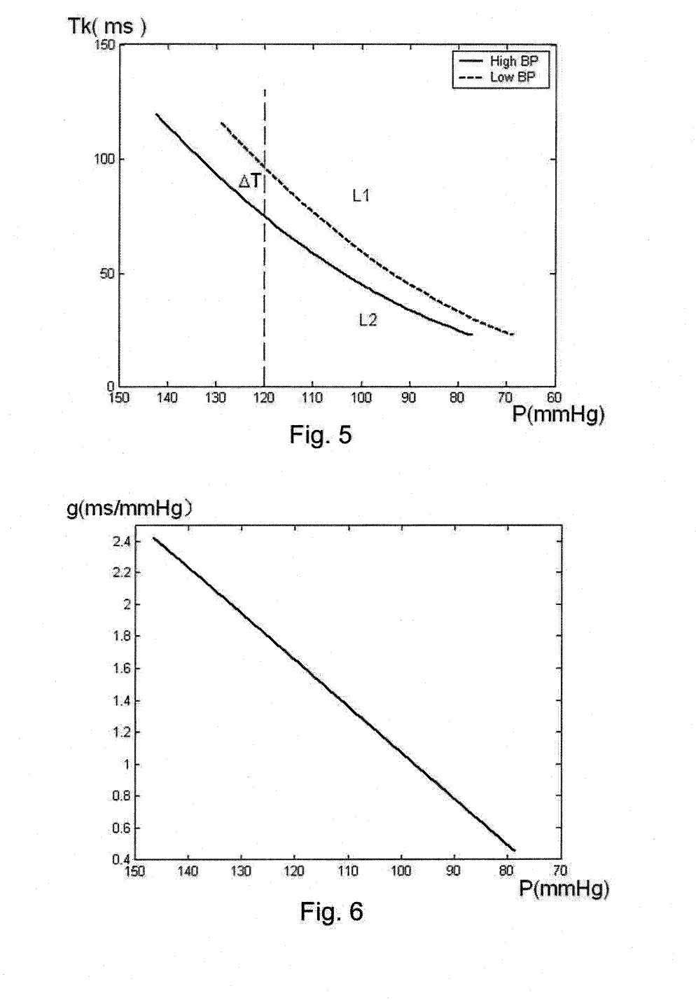

The following is the principle of said method: If the BP level when the T_{Km} is measured is equal to the BP level BP₀ when the function of T_{K}=H(P) was established, the obtained Korotkoff's sound delay time value T_{Km} should be equal to Korotkoff's sound delay time value T_{Km0} which is calculated from the fitted curve T_{K}=H(P) while the corresponding pressure is equal to Pₘ. Otherwise, it means that the BP has changed. If the blood pressure increases, the delay time value T_{Km} is shorter; and if the blood pressure decreases, the delay time value T_{Km} is longer (shown as FIG. 5). According to the phenomena that the change of Korotkoff's sound delay time induced by cuff pressure changes when blood pressure keeps stable is the same in size and inverse in direction of that induced by blood pressure changes when cuff pressure is stable, the BP change value when the T_{Km} is measured can be calculated.

### EMBODIMENT 2

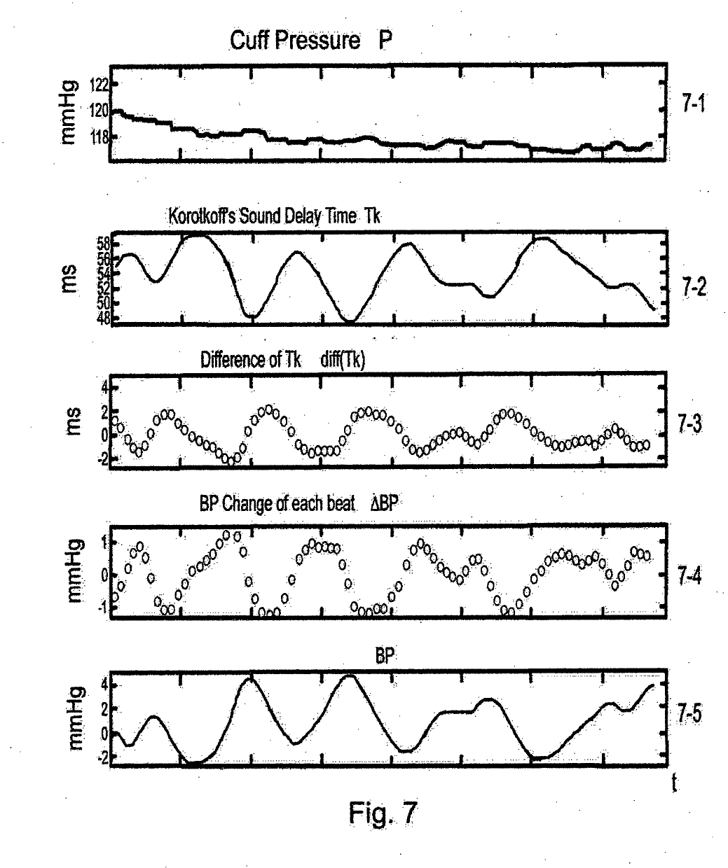

This is about how to measure beat-to-beat arterial blood pressure. FIG. 7 shows the course of data acquiring and signal processing which is shown in detail as the following:

1. Obtaining a series of Korotkoff's sound delay time values T_{K} and the corresponding cuff pressure values P during deflation of the cuff, and constructing the function of T_{K}=H(P) as shown in embodiment 1. After two curve fittings about these discrete data, establishing the curve of T_{K}=H(P) that Korotkoff's sound delay time value T_{K} changed with cuff pressure value P.

2. Calculating the difference value of said fitted curve T_{K}=H(P), and obtaining a new function of g(P) in which the Korotkoff's sound delay time changes with one unit pressure (1 mm Hg), as shown in FIG. 9.

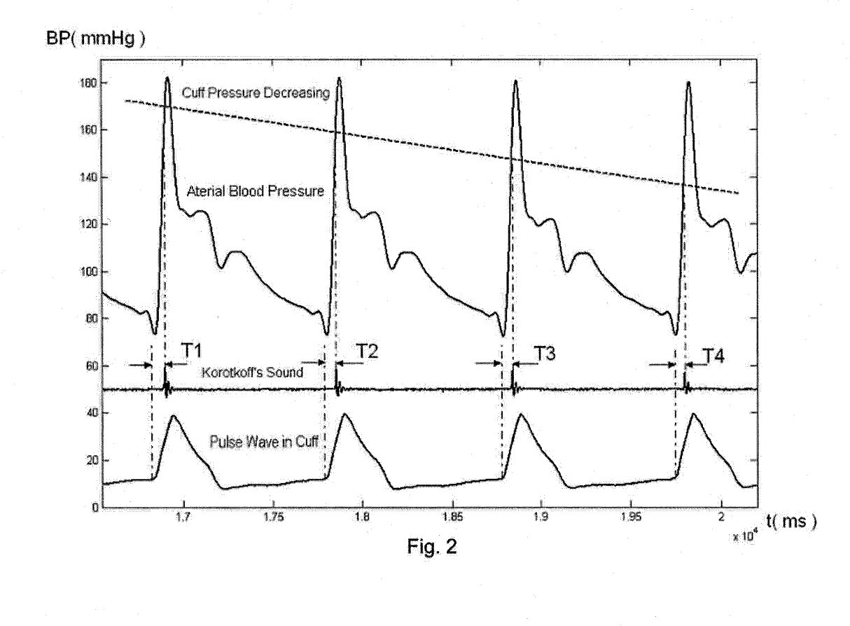

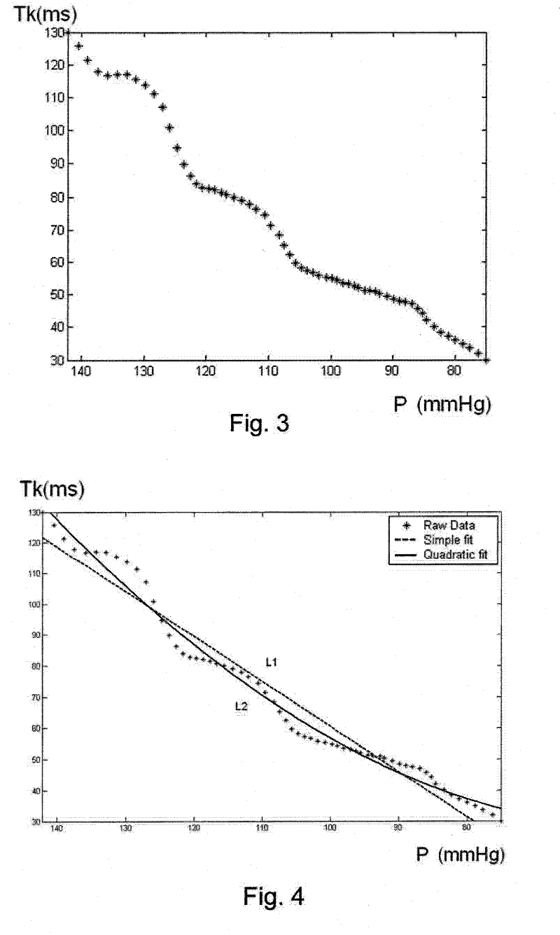

3. Maintaining the cuff pressure at the constant level P₀ that is approximately equal to a mean blood pressure which is between the systolic blood pressure SBP and the diastolic blood pressure DBP, and then getting a series of beat-to-beat Korotkoff's sound delay times T(i) (shown as FIG. 7-2).

4. Calculating the difference T′(i) of the said T(i) in the condition of the said approximate constant puff pressure P0 (shown as 7-3). The relationship is shown as the following equation.
T′(i)=T(i+1)−T(i) . . . (i=1,2,3 . . . )

5. Each T′(i) is corresponding to a known cuff pressure Pᵢ, and each pressure Pᵢ is corresponding to a unique datum, g(Pᵢ)=dT_{K}/dP. Therefore, calculating the dynamic BP change value ΔBP(i) of each beat by using the coefficient g(Pᵢ) corresponding to the T′(i) (shown as FIG. 7-4).
ΔBP(i)=T′(i)/g(Pᵢ)

Adding up ΔBP(i) of each beat, and obtaining the beat-to-beat continuous BP change value BP(n) (shown as FIG. 7-5):

$$BP ( n ) = {\sum }_{i = 1}^{n} Δ BP ( i )$$

wherein n=1 . . . m−1, m is the number of heartbeat cycles when the cuff pressure keeps approximate stable, and BP is the dynamic blood pressure. According to the above equation, the beat-to-beat BP change can be calculated.

The calculated arterial blood pressure change is more close to the actual status when the cuff pressure is at the level of mean blood pressure value or close to it.

### EMBODIMENT 3

This is an embodiment to implement individualized rectification by using the data of arterial blood pressure measurement method of this invention.

The regress equation of PWTT and beat-to-beat arterial blood pressure BP is:
BP=a+b×PWTT (A)

Wherein, BP is blood pressure, PWTT is pulse wave transmit time, and a and b are pending regress coefficients.

The method of individualized rectification of coefficient a and b is as the following:

(1) Putting the cuff and Korotkoff's sound sensor in the distal cuff on one of the upper arms of the subject, measuring blood pressure by an auscultatory method and getting the systolic blood pressure and diastolic blood pressure, calculating the mean arterial pressure BP₀ by empirical formula (which can also be accurately measured by oscillometric method), and recording the synchronous pulse wave transmit time (PWTT₀).

(2) Getting a series of pulse wave transmit times and the corresponding cuff pressures in the whole deflating process in the same way as in the embodiment 1, constructing the function of T_{K}(P); getting the curve of Korotkoff's sound delay time T_{K} changes with cuff pressure P (FIG. 8) by two curve fittings of these discrete data; calculating the difference of the above curve and getting the Korotkoff's sound delay times changes with each per unit pressure (1 mm Hg), and forming a new series of function of g(P). As shown in FIG. 9.

(3) Keeping the cuff pressure at a constant pressure between systolic blood pressure and diastolic blood pressure, getting a series of beat-to-beat Korotkoff's sound delay times and the corresponding pulse wave transmit times. In the measurement process, making the subject breathe deeply several times (e.g., 3 times), getting two groups of data arbitrarily, calculating the difference of Korotkoff's sound delay times at different times ΔT. Based on the series of functions of g(P) which is shown in item 2, calculating the value of g at the corresponding cuff pressure, using AT to estimate the change of arterial blood pressure ΔBP₁; calculating the change of the synchronous pulse wave transmit time ΔPWTT₁.

That is, getting the regressive coefficient b₁=ΔBP₁/ΔPWTT₁.

In the same way, getting the b₂, b₃ . . . separately based on several groups of data.

Calculating the average or median of the series b₁, b₂, b₃ . . . , thus the regressive coefficient can represent the actual individual parameters.

FIG. 10, from top to bottom, shows the estimated blood pressure changes and pulse wave transmit time PWTT changes based on the Korotkoff's sound delay times during a period of time, and the coefficient b of the blood pressure function which is calculated by the ratio of peak and trough. Calculating the average or median of the series b₁, b₂, b₃. . . , which is the final coefficient b.

In addition, you can also directly calculate the regressive coefficient by using the first BP signal and the second PWTT signal, and the regressive coefficient is the coefficient b of the blood pressure function.

FIG. 12 is the block diagram of the arterial blood pressure measurement apparatus of this embodiment.

In this embodiment, the controlling ports of inflating and deflating units connect to a CPU, which controls the inflating and deflating. The analog signals output from the cuff pressure sensor are amplified, low-pass and band-pass filtered, and converted to digital signals by an A/D converter and input to the CPU; the output signals of the Korotkoff's sound sensor are amplified, filtered, converted to digital signals by an A/D converter and input to the CPU; electrocardiogram circuits connect electrodes and the CPU.

What is claimed is:

1. A method of arterial blood pressure measurement, characterized in that said method comprises:
(1) wrapping the cuff around the limbs of a given subject, obtaining a series of cuff pressure values P and the corresponding Korotkoff's sound delay time values T_{K}, A function of T_{K}=H(P) that describes the relationship between the Korotkoff's sound delay time T_{K} and the cuff pressure P can be obtained;
(2) according to the phenomena that the change of cuff pressure can cause Korotkoff's sound delay time alter at a constant blood pressure and the change of blood pressure can cause Korotkoff's sound delay time alter at a constant cuff pressure, and combined with the Korotkoff's sound delay time (Tₖₘ) in the distal to the cuff at a certain cuff pressure Pₘ and said function of T_{K}=H(P) between Korotkoff's sound delay time T_{K} and cuff pressure P, the change of the blood pressure values corresponding to the measured Korotkoff's sound can be estimated.

2. The method of arterial blood pressure measurement according to claim 1, characterized in that said method comprises:
Obtaining function of T_{K}=H(P) by a series of Korotkoff's sound delay time value and the corresponding cuff pressure values in course of deflation of the cuff;
Curve fitting of the discrete data by using simple line approximation or quadratic line approximation, getting a curve which shows the relation between the Korotkoff's sound delay time and the cuff pressure;
According to the function of T_{K}=H(P) , getting a new function of g(P)=dT_{K}/dP, g(P) is also a function of cuff pressure P and g is the ratio of Korotkoff's sound delay time changes of each point and cuff pressure changes;
Converting the change of the Korotkoff's sound delay time T_{K} at a certain cuff pressure to the change of arterial blood pressure P.

3. The method of arterial blood pressure measurement according to claim 1, characterized in that said method comprises:
After getting the Korotkoff's sound delay time T_{Km} at a certain cuff pressure Pₘ, according to the phenomenon that the change of Korotkoff's sound delay time induced by cuff pressure change when blood pressure is stable is the same in size and inverse in direction of that induced by blood pressure change when cuff pressure is stable and according to the function of T_{K}=H(P) which shows the changes of Korotkoff's sound delay time with the cuff pressure, estimating the change of arterial blood pressure corresponding to the detected Korotkoff's sound delay time.

4. The method of arterial blood pressure measurement according to claim 3, characterized in that said method comprises:
After getting the Korotkoff's sound delay time T_{Km} corresponding to a certain cuff pressure Pₘ which is between systolic pressure and diastolic pressure, according to the Korotkoff's sound delay time T_{Km0} when cuff pressure is Pₘ in said function of T_{K}=H(P), getting the difference ΔT_{Km} of T_{Km} and T_{Km0};
According to the function of g(P)=dT_{K}/dP, getting the g(Pₘ) when cuff pressure is Pₘ;
According to g(Pₘ)=ΔT_{Km}/ΔBPₘ, getting the change of blood pressure ΔBPₘ corresponding to the measured Korotkoff's sound delay time, and ΔBPₘ plus the mean blood pressure BP₀ when the function is established, the sum is the current blood pressure of this beat.

5. The method of arterial blood pressure measurement according to claim 3 characterized in that said method comprises:
when measures the Korotkoff's sound delay time T_{Km} at a certain cuff pressure Pₘ, keeping the cuff pressure at an approximate constant value which is between the systolic blood pressure and the diastolic blood pressure, and measuring the serial of Korotkoff's sound delay time T(i) for each heart beat;
Differentiating T(i) and the difference serial is T′(i);
According to said function of T_{K}=H(P), the ratio of the change of the Korotkoff's sound delay time dT_{K} and the change of the cuff pressure dP is a function of cuff pressure P, which is g(Pₘ), the blood pressure change of each beat can be estimated
ΔBP(i)=T′(i)/g(Pᵢ)
Accumulating the ΔBP(i) of each beat, and getting the continuous beat-to-beat blood pressure change:
$$BP ( n ) = {\sum }_{i = 1}^{n} Δ BP ( i )$$
wherein n=1 . . . m−1, m is the number of heartbeat cycle when the cuff pressure keeps approximate stable, BP is the dynamic blood pressure.

6. Individualized rectifying technology by using the method of arterial blood pressure measurement in claim 1, said technology comprises: to construct the regress equation between beat-to-beat blood pressure BP and pulse wave transit time PWTT for the a given subject:
BP=a+b×PWTT (A)
wherein BP is arterial blood pressure, PWTT is the pulse wave transit time of corresponding to the arterial blood pressure, b is regress coefficient and a is another equation coefficient;
Having rectified the equation coefficients of a and b for a specific individual, applying the equation and the continuous measurement of pulse wave transit time, the blood pressure can be continuously monitored, characterized in that the method of individualized rectifying technology for regress coefficient b comprises:
(1) wrapping the cuff around the limbs of a given subject, obtaining a series of cuff pressure values and the corresponding Korotkoff's sound delay time values, a function of T_{K}=H(P) between the Korotkoff's sound delay time and the cuff pressure can be obtained;
(2) measuring the Korotkoff's sound delay time T_{Km} at a certain cuff pressure Pₘ, and applying the function of T_{K}=H(P) between Korotkoff's sound delay time and cuff pressure to estimate blood pressure change of the corresponding detected Korotkoff's sound delay time;
(3) recording the pulse wave transit time corresponding to the Korotkoff's sound delay time in item (2);
(4) applying the data in the item (2) and (3) to calculate the ratio of mean arterial blood pressure change and the corresponding pulse wave transit time change, the ratio is the individualized rectifying coefficient b.

7. The individualized rectifying technology according to claim 6, characterized in that said technology comprises:
when measure Korotkoff's sound delay time at a certain cuff pressure Pₘ, said cuff pressure is the value which is equal to or approximate to the mean arterial pressure.

8. The individualized rectifying technology according to claim 6, characterized in that said technology comprises:
when measure Korotkoff's sound delay time at a certain cuff pressure Pm, apply the act which can alter the blood pressure and don't alter the characteristic of the blood vessel wall to enhance the range of blood pressure change between different measurement points.

9. The individualized rectify technology according to claim 8, characterized in that said technology comprises:
said act that can alter the blood pressure is deep respiratory movement.

10. The apparatus for arterial blood pressure measurement used by the method of claim 1, characterized in that said apparatus comprises:
a cuff which has inflating unit, deflating unit and cuff pressure sensor and a Korotkoff's sensor and ECG electrode;
wherein the output signal ports of the said cuff pressure sensor and Korotkoff's sound sensor connect to the microprocessor through the signal conditioning circuit; and said ECG electrode connects to microprocessor via ECG circuit;
and said microprocessor has data display device and/or print output device.
